(19) United States
(12) Patent Application Publication
(10) Pub. No.: US 2025/0235197 A1
(43) Pub. Date: Jul. 24, 2025

(54) SUTURE RETENTION SYSTEM AND METHOD

(71) Applicant: Riverpoint Medical, LLC, Portland, OR (US)

(72) Inventors: William McKinley White, III, Portland, OR (US); Elliot Bixby, Milwaukie, OR (US)

(21) Appl. No.: 19/036,779

(22) Filed: Jan. 24, 2025

Related U.S. Application Data
(60) Provisional application No. 63/624,515, filed on Jan. 24, 2024.

(51) Int. Cl. A61B 17/04

(52) U.S. Cl. CPC A61B 17/0401; A61B 2017/0409

(57) ABSTRACT

The invention relates to a suture retention system or kit for attachment of tissue-to-bone-to-implant, tissue-to-implant, or bone-to-implant. The suture retention system or kit includes a plurality of sutures, a plug ball, and a needle that can facilitate retention and attachment of the implant prosthesis and other tissue during fixation. The plug ball or structure is a soft, flexible, three-dimensional structure that has a resident volume. In one embodiment, upon tensioning of the plurality of sutures during deployment, the plug ball or structure shortens axially and expands radially, achieving a larger diameter than the hole or opening through which it was placed, thus resisting pull out and achieving retention, as well as reduce implant prosthesis migration.

### CROSS-REFERENCE TO RELATED APPLICATIONS

This application claims the benefit of U.S. Provisional Application No. 63/624,515 entitled “Suture Retention System and Methods” filed on Jan. 24, 2024, the disclosure of which is incorporated by reference herein in its entirety.

### TECHNICAL FIELD

The invention generally relates to suture retention systems that can be used to secure tissues, bone, or muscles to one or more prosthetic implants. More specifically, the embodiments described herein relate suture retention systems and methods for securing at least a portion of the prosthetic implants to the humeral stem implant and/or to the humerus and the subscapularis.

### BACKGROUND OF THE INVENTION

Total shoulder replacement, also known as total shoulder arthroplasty (TSA), is a procedure where a damaged or diseased shoulder ball and socket joint are replaced with a prosthesis. In TSA procedures, the subscapularis is distracted and the humeral articular surface is replaced with a humeral implant. To reattach the subscapularis or fracture fragments back onto the humerus repaired with the humeral implant, sutures are passed through suture holes in the stemmed humeral implant and through holes drilled laterally into the humerus or around the bone fragments. However, the threading of sutures through suture holes of the stemmed humeral implant can be tedious, cumbersome and time consuming. Additionally, given the complexity of the anatomy and biomechanics of the shoulder joint, when two or more separate sutures are used to secure the subscapularis or fracture fragments to the stemmed humeral implant, the first suture tightened down will have a tendency to shift or require further tensioning after the second suture has been tightened down. This results in a surgeon having to repeat the tensioning process multiple times on at least one suture to reach an optimal level of tension across all the sutures used in the procedure.

### BRIEF DESCRIPTION OF THE SEVERAL VIEWS OF THE DRAWINGS

FIG. 1 depicts a side view of a first embodiment of a suture retention system;

FIG. 2A depicts an isometric view of the suture retention assembly of FIG. 1;

FIGS. 2B-2D depict a side view of the suture retention assembly of FIG. 2A with different suture plug sizes;

FIGS. 3A-30 depicts one embodiment of a method of making a suture retention assembly;

FIG. 4 depicts a second embodiment of a suture retention assembly;

FIG. 5 depicts a third embodiment of a suture retention assembly;

FIG. 6 depicts a fourth embodiment of a suture retention assembly;

FIGS. 7A-7C depicts top views of one embodiment of a suture retention assembly positioning during a method of use;

FIGS. 8A-8C depicts top views of a second embodiment of using a suture retention assembly to attach to bone;

FIGS. 9A-9D depicts top views of a third embodiment of using a suture retention assembly to attach to bone;

FIGS. 10A-10D depicts top views of a fourth embodiment of using a suture retention assembly to attach to bone;

FIGS. 11A-11D depicts one embodiment of a method of fixating one or more suture retention assemblies to bone; and

FIGS. 12A-12G depicts an alternate embodiment of a method of fixating one or more suture retention assemblies to bone.

### BRIEF SUMMARY OF INVENTION

In one exemplary embodiment, the suture retention system or kit comprises at least one suture retention assembly. The suture retention assembly comprises a first suture, at least one plug or occluder and a suture needle. The at least one suture comprises a first end and a second end. The at least one plug positioned between the first end and the second end of the at least one suture. At least a portion of the first end and the second end of the at least one suture coupled to the suture needle. The suture retention assembly further comprises a second suture. The plug may be sliding or non-sliding. The suture retention system may further comprise an implant prosthesis and/or one or more anchoring elements.

In another embodiment, the suture retention system or kit comprises at least one suture retention assembly. The suture retention assembly comprises a first suture, a second suture, at least one plug or occluder and a suture needle. The first and second suture comprises a first end and a second end. The at least one plug positioned between the first end and the second end of the first and second sutures. At least a portion of the first end and the second end of first and second sutures are coupled to the suture needle. The suture retention assembly further comprises a second suture. The suture retention system may further comprise an implant prosthesis and/or one or more anchoring elements.

In another embodiment, the suture retention system or kit comprises a suture retention assembly and an implant prosthesis. The suture retention system may further comprise one or more anchoring elements. The suture retention assembly comprises a first suture, at least one plug or occluder and a suture needle. The at least one suture comprises a first end and a second end. The at least one plug positioned between the first end and the second end of the at least one suture. At least a portion of the first end and the second end of the at least one suture coupled to the suture needle. The suture retention assembly further comprises a second suture. The anchoring element further comprises a flexible anchor or a rigid or hard anchor. The anchoring element may comprise an anchoring material, the anchoring material may include ceramic, polymer or metal. The implantable prosthesis may be comprise any prosthesis used for a variety of orthopedic applications. More specifically, the implantable prosthesis may be used for repairing a bone or bone surface, such as a humerus (upper arm bone), a femur (thigh bone), or any other suitable bone. In one embodiment, the implantable prosthesis may include a base head (stemless). The implantable prosthesis may include a base head and a stem extending from the base head along a longitudinal axis from a proximal end portion to a distal end portion. The stem and/or base head is configured to be inserted into a bore that has been reamed into or otherwise produced within a bone (e.g., the humerus).

### DETAILED DESCRIPTION OF THE INVENTION

In accordance with the embodiments disclosed herein, there is a need for an improved suture retention system or kit and its respective technique for tensioning and securing the tissue-to-implant-to-bone. More specifically, there is a need for an improved suture retention system or kit and its respective technique for tensioning and securing the subscapularis-to-implant-to-humerus. Furthermore, a need exists to develop a new technique that can be performed with proficiency by a full spectrum of orthopedic surgeons who presently struggle with existing suture tensioning techniques.

The suture retention system or kit is provided to help the physician with a firm, reliable fixation implant relative to the bone or tissue surface throughout the healing period. The suture retention system comprises a suture retention assembly that when deployed into a pilot hole, channel, or opening on a surface of an implant prothesis, the plug engages with the pilot hole or opening to wedge or lock into place to create an anchoring point at an anchoring location, allowing the first and second ends to exit out a pilot hole for future fixation to the bone and/or tissue. At least a portion of the plug may engage with a top surface of the pilot hole and/or within the inner diameter of the pilot hole.

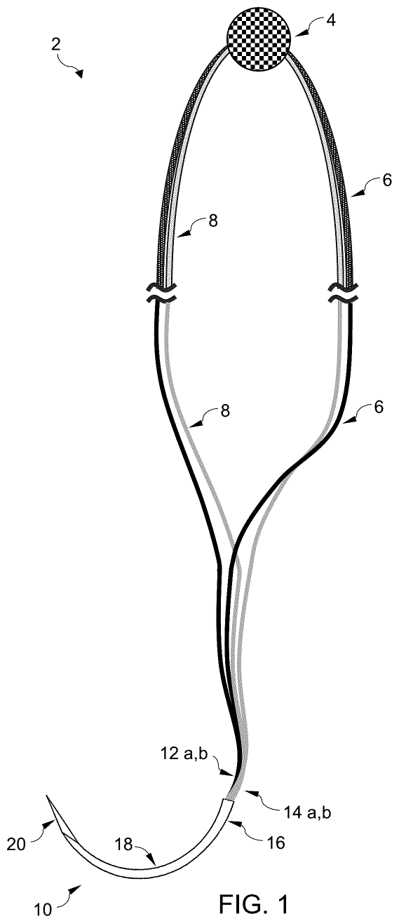

FIG. 1 depicts a side view of a first embodiment of a suture retention assembly. The suture retention assembly 2 comprises at least one plug or occluder 4, a first suture 6, a second suture 8, and a suture needle 10. The first suture 6 comprises a first end 12a and a second end 12b, and the second suture 8 comprises a first end 14a and a second end 14b. The at least one plug 4 positioned between the first end 12a, 12b and the second end 14a, 14b of the first suture 6 and second suture 8. At least a portion of the first suture 6 and the second suture 8 extending through a portion of the plug 4. Accordingly, at least a portion of the first suture 6 and the second suture 8 extending through a lumen with the plug 4.

The first end 12a, 14a and the second ends 12b, 14b of the first suture 6 and second suture 8 coupled to the suture needle 10. Coupling may comprise swaging, adhesive, or any other method known in the art. The plug or occluder 4 may be movable or slidable relative to the first suture 6 and the second suture 8. Alternatively, the plug or occluder 4 may be fixed relative to the first suture 6 and the second suture 8. The plug or occluder 4 may be also known as plug ball or plug wad.

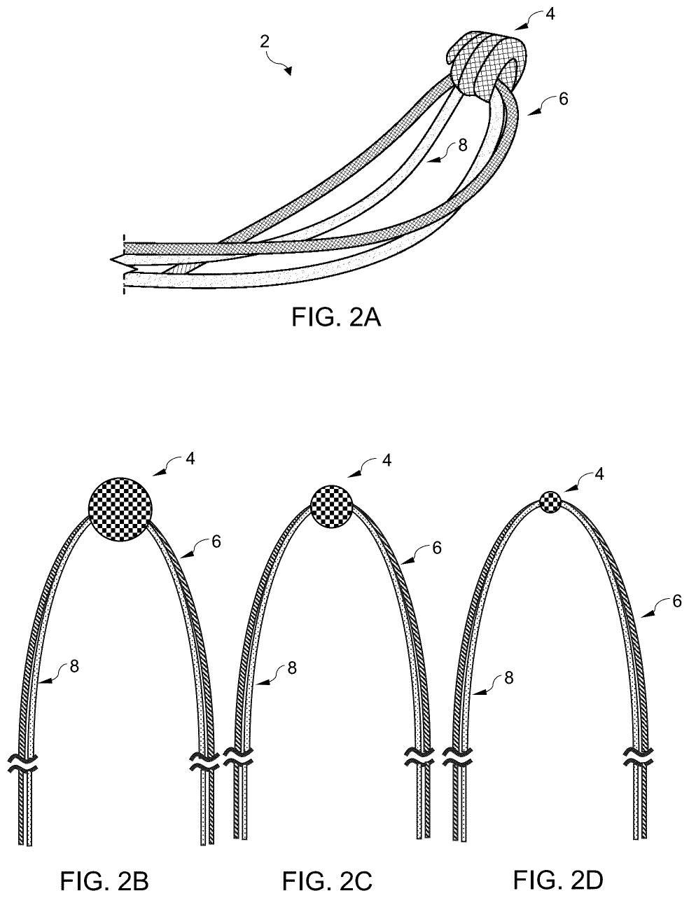

FIGS. 2A-2D depicts different views of different sizes of a plug or occluder 4. The plug or occluder 4 may comprise a flexible plug or a rigid plug. The plug or occluder 4 may comprise different sizes, the different sizes may include small, medium, large, x-large and/or any combination thereof. The different sizes may be manufactured to accommodate the intended pilot hole size. The flexible plug may comprise a textile plug.

In one embodiment, the plug 4, plug ball or occlude may, in some embodiments, be constructed in a manner that it creates a three-dimensional structure, such as a ball. Upon the suture retention assembly 2 being deployed, the soft three-dimensional plug 4 may expand, or be compelled to expand from an uncompressed state to a compressed state. More specifically, when the first end 12a, 14a and second free end 12b, 14b of the first suture 6 and second suture 8 are tensioned or pulled, the plug 4 collapses or deforms from a smaller shape to a larger shape that does not fit back through a pilot hole, opening or channel through which it was introduced, thus achieving the anchoring or retention required. Accordingly, when the suture free ends 12a, 12b, 14a, 14b are tensioned with some counter traction against a top surface of the implant prosthesis, bone surface and/or pilot hole inner diameter, the plug 4 compresses axially and expands radially. In one exemplary embodiment, the plug 4 may change from a first uncompressed position or state to a compressed position or state. Alternatively, the plug or plug ball 4 may comprise a uniform shape that does not change from an uncompressed state to a compressed state.

The plug 4, the first suture 6 and/or the second suture 6 comprise a suture material. The suture material may comprise absorbable or non-absorbable materials. The suture materials may comprise synthetic or natural materials. The suture materials may comprise a polymer or metal material. The absorbable materials may comprise polyglycolic acid, polyglactin, polyglactin fast, catgut chromic, catgut plain, poliglecaprone, polydioxnone, and/or any combination thereof. The non-absorbable materials may comprise polypropylene, Nylon, silk, polyester, polyethylene, ultra-high weight molecular polyethylene, polytetrafluoroethylene, and/or any combination thereof. The metal materials may include stainless steel, nitinol, aluminum, silver, copper and/or any combination thereof.

The plug 4, the first suture 6 and/or the second suture 8 may comprise monofilament or multifilament sutures. The multifilament sutures may comprise a textile. The textile includes woven, knitted and/or braided textile. The plug 4, the first suture 6 and the second suture 8 may include a flat suture, a round suture, a flat-to-round suture, and/or any combinations thereof. The plug 4, the first suture 6 and the second suture 8 may comprise cored or coreless sutures. In one exemplary embodiment, the plug comprises a coreless UHMWPE braided textile.

In one embodiment, the plug 4, the first suture 6 and/or the second suture 8 may comprise a braided textile, the braided textile may include a plurality of carriers. The carriers may comprise 10 to 30 carrier; the carriers may comprise 10 to 25 carriers; the carriers may comprise 10 to 20 carriers; the carriers may comprise 15 to 18 carriers; the carriers may comprise at least 15 carriers or greater; and/or the carriers may comprise at least 20 carriers or less.

In another embodiment, the plug 4, the first suture 6 and the second suture 8 may comprise a denier. The denier may comprise 75 denier to 125 denier; the denier may comprise 75 denier to 115 denier; the denier may comprise 75 denier to 105 denier; the denier may comprise at least 75 denier or greater; the denier may comprise at least 90 denier or greater; and/or the denier may comprise at least 100 denier or greater.

In another embodiment, the plug 4, the first suture 6 and the second suture 8 may comprise a picks per inch (PPI). The PPI may comprise 15 PPI to 40 PPI; the PPI may comprise 15 PPI to 35 PPI; the PPI may comprise 15 PPI to 30 PPI; the PPI may comprise 20 PPI to 30 PPI; the PPI may comprise 25 PPI to 30 PPI; the PPI may comprise at least 25 PPI or greater. Accordingly, the first suture 6 may comprise a first pattern and a first color, and the second suture 8 may comprise a second pattern and a second color. The first suture pattern may comprise a same or different pattern than the second suture pattern. The first suture color may comprise a same or different color than the second suture color.

The needle or suture needle 10 comprises a swaged end 16, a needle body 18 and a needle point 20. The needle 10 should be rigid enough to resist distortion while being inserted through cortical bone, yet flexible enough to bend before breaking, be as slim as possible to minimize trauma, sharp enough to penetrate tissue with minimal resistance, and be stable within a needle holder to permit accurate placement. The free ends 12a, 12b, 14a, 14b of the first suture 6 and the second suture 8 are coupled to the swaged end 16 of the needle 10 The needle body or shaft 18 is the region grasped by the needle holder and/or a surgical instrument (e.g., forceps). The needle body 18 can be round, cutting, or reverse cutting. The needle body 18 including a curved or arched body, the curved or ached body comprises a curvature and a curvature radius. The needle body 18 may comprise a straight body, ½ curved body, a compound curved body, a J-shape body, a ⅝ circle body, a ¼ circle body, a ⅜ circle body, a ½ circle body. The curvature radius includes ¼, ⅜, ½, and/or ⅝. The needle point 20 acts to pierce the tissue or cancellous bone and can be either conventional cutting, tapered, tapered cutting, curved cutting, reverse cutting, micropoint reverse cutting, reverse cutting prime, blunt and/or any combination thereof.

In another embodiment, the suture retention system or kit further comprises one or more anchoring elements. The anchoring element further comprises a flexible anchor or a rigid or hard anchor. The anchoring element may comprise one or more bone anchors or buttons. The bone anchors may include a screw-in suture anchor, and interference fit suture anchor, knotted suture anchors, knotless suture anchors, a fixation screw, a textile anchor, and/or any combination thereof. The anchoring element may further comprise an all-suture anchor, a metal anchor and/or a biocomposite anchor. The anchoring element may comprise an absorbable or non-absorbable anchor. The anchoring element may comprise a button, a screw-in and non-screw-in design. A non-screw-in anchor is an anchor that toggles, pushes in, or changes their morphology after being pushed into the bone.

### Methods of Making Suture Retention Assembly

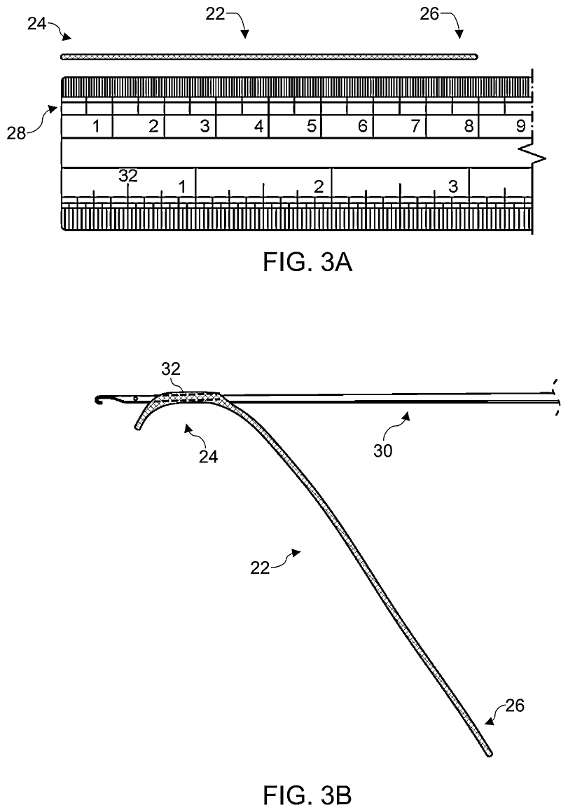

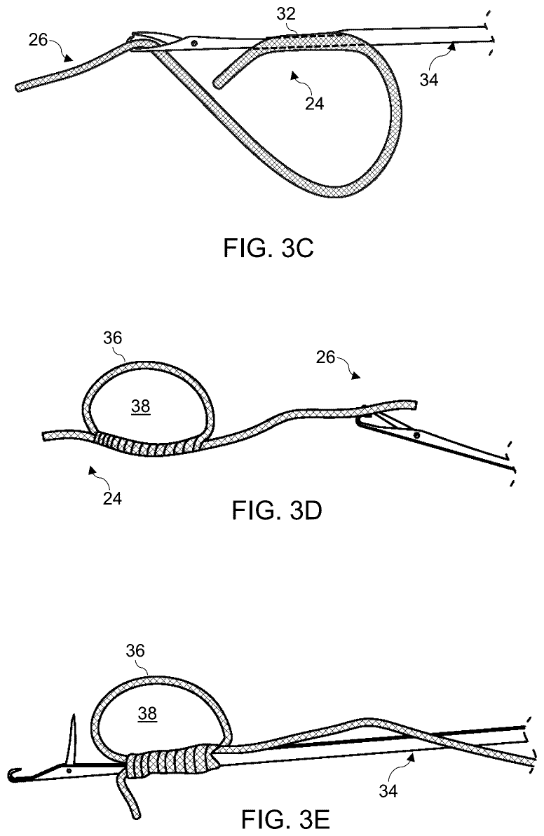

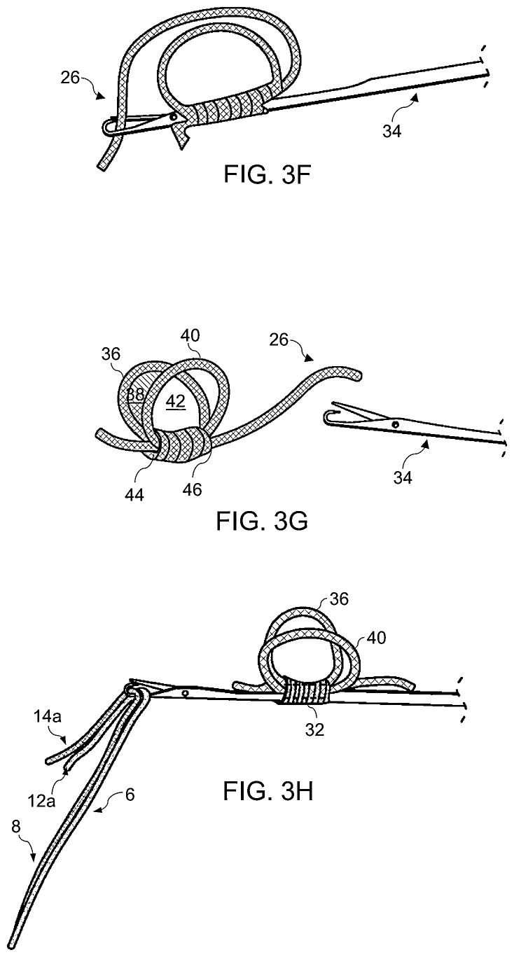

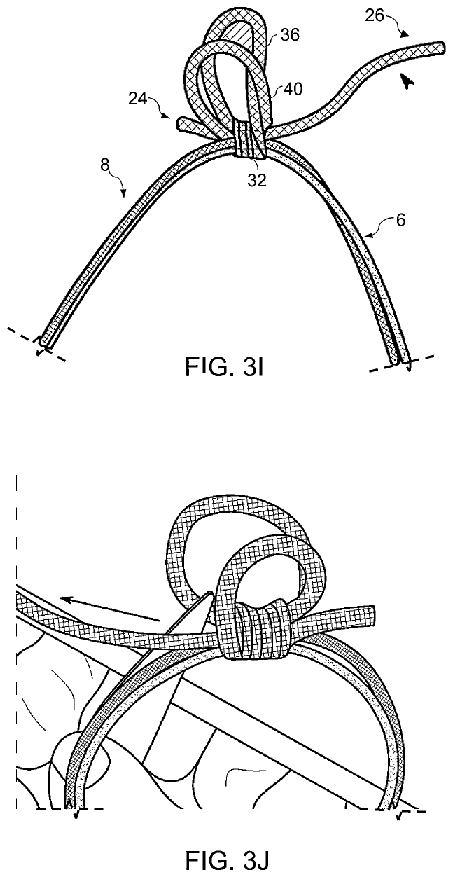

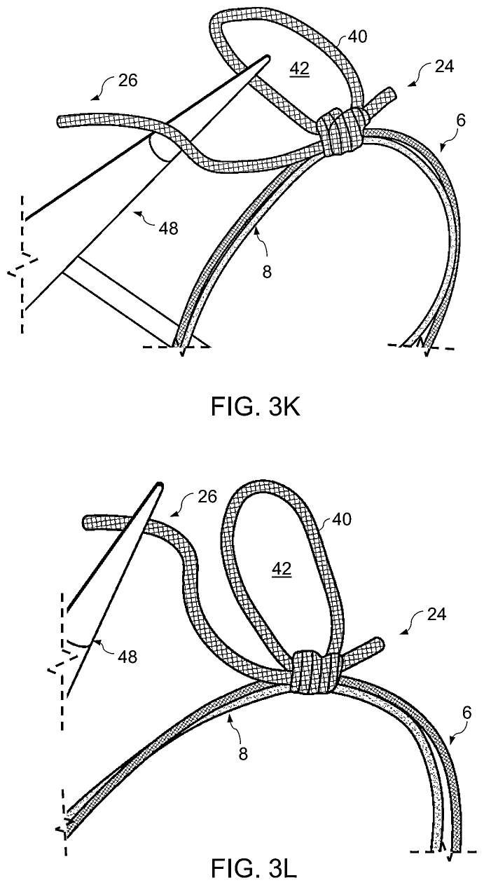

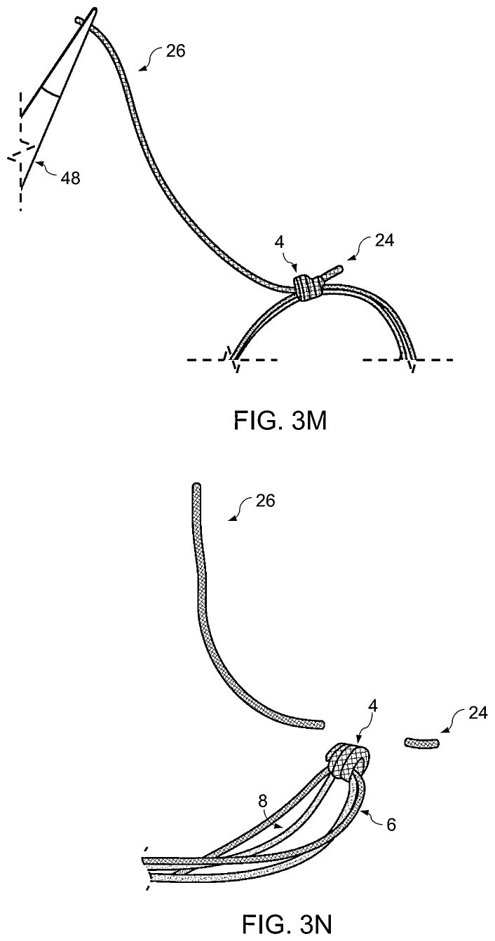

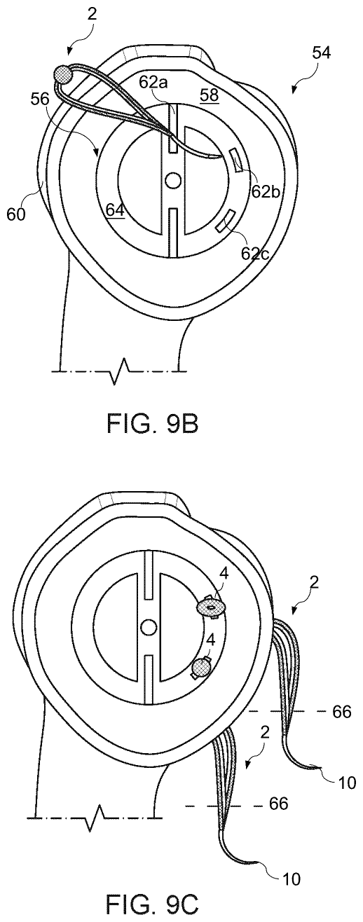

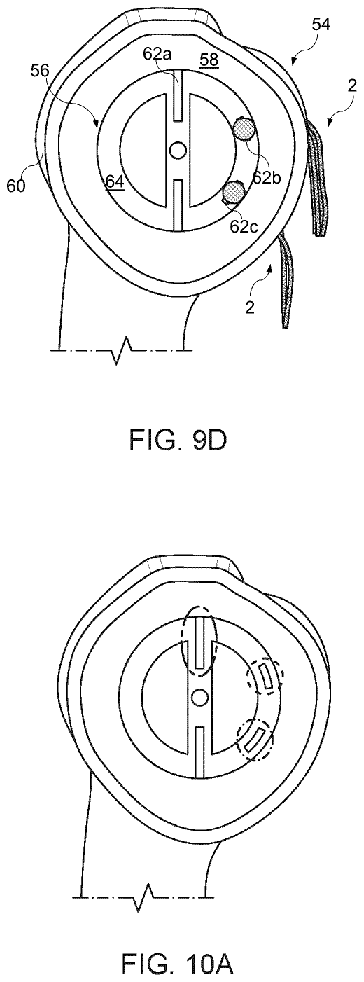

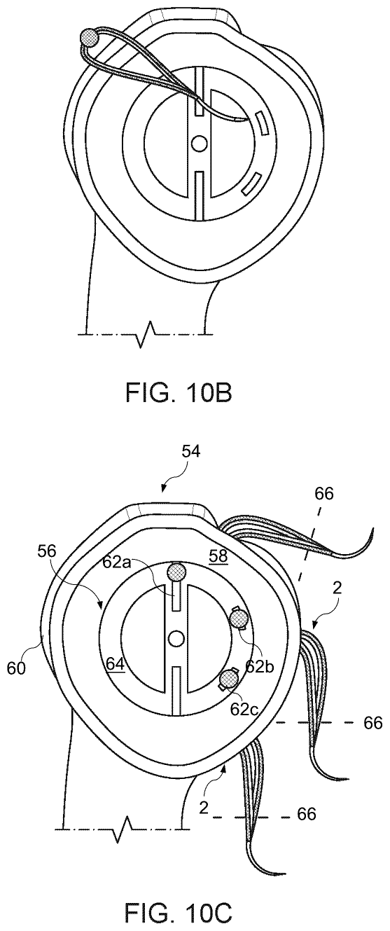

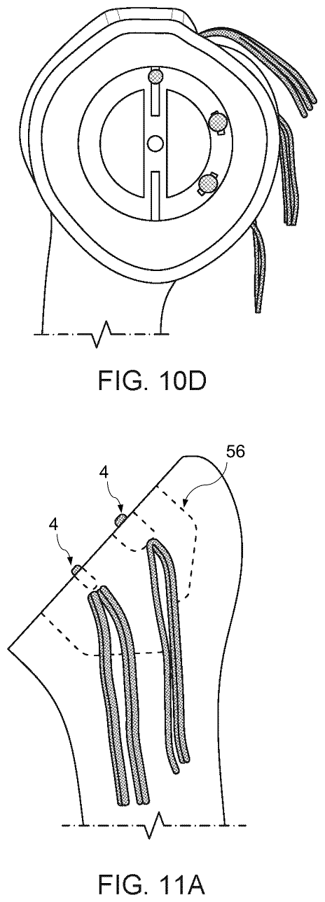

FIGS. 3A-30 depict several plan views of depicts one embodiment of a method of making a suture retention assembly. The method of making a suture retention assembly comprises the step of: obtaining a first suture 6, a second suture, and a plug suture 22. The plug suture may comprise a suture length, the suture length may include at least 5 cm; the suture length may include at least 8 cm; the suture length may include at least 10 cm; the suture length may include a range of 5 cm to 10 cm; and/or the suture length may include a range of 7 cm to 9 cm. The plug suture 22 comprises a first tail end 24 and a second tail end 26. In one exemplary embodiment the plug suture 22 may comprise a material and a textile type, the material includes a coreless UHMWPE braided suture and the textile type includes a braided textile.

At least a portion of the first tail end 24 of the plug suture 22 may be pierced with a tool 30 to create a lumen 32. In one exemplary embodiment, the piercing may occur substantially at 1.5 cm from the first tail end 24 of the plug suture 22 and the tool 30, 34 may exit the lumen 32 after 1 cm from the first tail end 24 of the plug suture 22. The lumen 32 may comprise a lumen length of 0.25 cm to 1 cm; the lumen length may comprise a range of 0.25 cm to 0.75 cm; the lumen length may comprise a range of 0.5 cm to 0.75 cm; the lumen length may comprise at least 0.25 cm and/or the lumen length may comprise at least 0.5 cm.

At least a portion of the second tail end 26 of the plug suture 22 extends through the lumen 32 using another tool 30, 34 to create a first looped segment 36 and a first loop opening 38. At least a portion of the second tail end 26 of the plug suture 22 extends through the lumen 32 using a tool 30, 34 to create a second looped segment 40 and a second loop opening 42. Pulling on the second tail end 26 of the plug suture 22 may help reduce the size of the first loop opening 38 and/or the second loop opening 42, thus creating a preformed plug. At least a portion of the first tail end 24 extends from a first lumen opening 44 and the second tail end 26 extends from the second lumen opening 46. The first tail end 24 may be diametrically opposed to the second tail end 26.

At least a portion of the first free end 12a, 14a of the first suture 6 and the second free end of the second suture 8, extends through the lumen 32. The plug suture 22, the first looped segment 36 and the second looped segment 40 comprises a preformed plug. The preformed plug and/or the plug 4 is positioned between the first free end 12a, 14a of the first suture 6 and the second free end of the second suture 8. Alternatively, the preformed plug and/or the plug 4 is centered between the first free end 12a, 14a of the first suture 6 and the second free end of the second suture 8.

At least a portion of the second tail end 26 of the plug suture 22 can be tensioned to reduce the size of the first loop opening 38. A tool 48 (e.g., pliers) may be used to hold the second loop segment 22 while at least a portion of the second tail end of the plug suture 22 is tensioned or pulled in order to maintain (and not reduce) the second loop opening 42 of the second looped segment 40. The reduction of the first loop opening 38 size allows a portion of the first looped segment 36 to contact a portion of the outer surface of the plug suture 22 and/or the lumen 32. At least a portion of the second tail end 26 of the plug suture 22 can be tensioned again to reduce the size of the second loop opening 42. The reduction of the second loop opening 42 size allows a portion of the second looped segment 40 to contact a portion of the outer surface of the plug suture 22, the first looped segment 36 and/or the lumen 32. Reducing both the first loop opening 38 and the second loop opening 42 creates the suture plug 4. At least a portion of the first tail end 24 and the second tail end 26 should be severed to create a compact suture plug 4. The suture plug 4 may be fixed or movable. If the suture plug 4 is movable, it is movable relative to the first suture 6 and the second suture 8.

FIG. 4 depicts an isometric view of a second embodiment of a method of making a suture retention assembly 2. In this embodiment, the suture retention assembly 2 comprises a suture plug 4 that is non-movable or fixed relative to the first suture 6 and the second suture 8. The method of making a suture retention assembly may comprise the steps discussed in FIGS. 3A-3N. However, the method of making a suture retention assembly 2 may further comprise the step of inserting or extending 48 the first free ends 12a, 14a and/or the second free ends 12b, 14b of the first suture 6 and the second suture 8 through the first loop opening 38 and the second loop opening 42.

At least a portion of the second tail end 26 of the plug suture 22 can be tensioned to reduce the size of the first loop opening 38 as described in FIG. 3J. A tool 48 (e.g., pliers) may be used to hold the second loop segment 22 while at least a portion of the second tail end of the plug suture 22 is tensioned or pulled in order to maintain (and not reduce) the second loop opening 42 of the second looped segment 40. The reduction of the first loop opening 38 size allows a portion of the first looped segment 36 to contact a portion of the outer surface of the plug suture 22 and/or the lumen 32. At least a portion of the second tail end 26 of the plug suture 22 can be tensioned again to reduce the size of the second loop opening 42. The reduction of the second loop opening 42 size allows a portion of the second looped segment 40 to contact a portion of the outer surface of the plug suture 22, the first looped segment 36 and/or the lumen 32. Reducing both the first loop opening 38 and the second loop opening 42 creates the suture plug 4. At least a portion of the first tail end 24 and the second tail end 26 should be severed to create a compact suture plug 4.

In one embodiment, the method of making a suture retention assembly 2 comprises the steps of: obtaining a first suture, a second suture, and a plug suture, the plug suture comprises a first tail and a second tail end; piercing the first tail end through the first tail end longitudinally at least two times to create at least two looped segments proximate to each other and a lumen; extending a first or second free end of the first and second suture through the lumen of the plug suture longitudinally; extending the first tail end and/or second tail end through the openings of the at least two looped segments; tensioning the first tail end and/or second tail end of the plug suture to close or reduce the diameter of the at least two looped segments to form a plug, a plug wad or plug ball; trimming the first tail and second tail end. The method of making the suture retention assembly further comprises coupling the first end and the second ends of each of the first and second sutures to a suture needle.

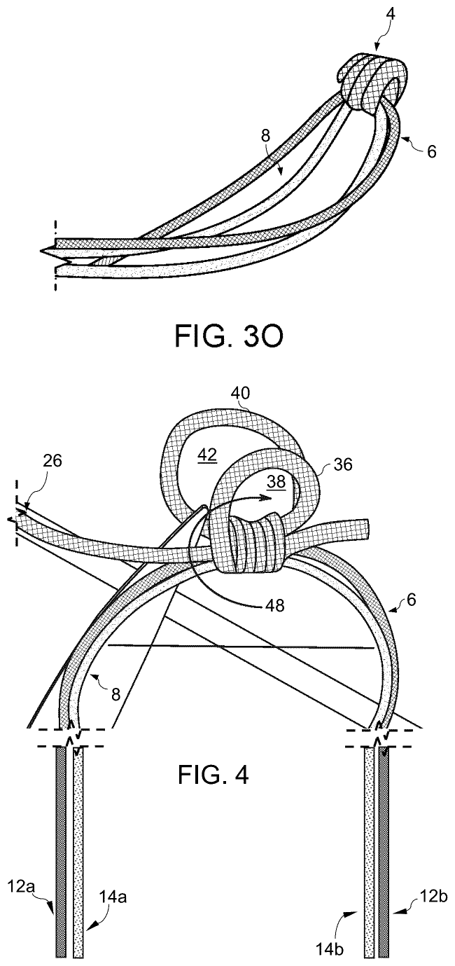

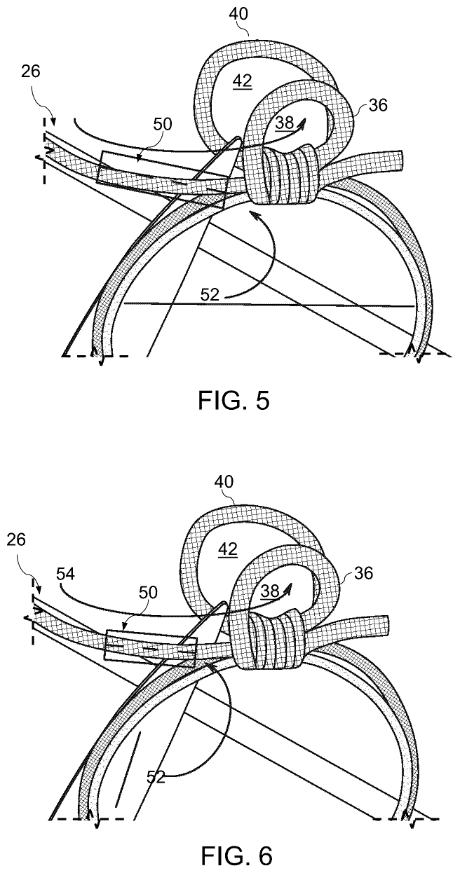

FIGS. 5 and 6 depicts an isometric view of a third and fourth embodiment of a method of making a suture retention assembly 2. In this embodiment, the suture retention assembly 2 comprises a suture plug 4 that is non-movable or fixed relative to the first suture 6 and the second suture 8. The method of making a suture retention assembly may comprise the steps discussed in FIGS. 3A-3N. However, the method of making a suture retention assembly 2 may further comprise the steps of: splicing or extending 52 the first the first free ends 12a, 14a and/or the second free ends 12b, 14b of the first suture 6 and the second suture 8 through a second lumen 50 positioned within or adjacent to the first tail end 24 of the plug suture and/or the second tail end of the plug suture 26; and inserting or extending 52 the spliced first free ends 12a, 14a and/or the second free ends 12b, 14b of the first suture 6 and the second suture 8 through the first loop opening 38 and/or the second loop opening 42.

At least a portion of the second tail end 26 of the plug suture 22 can be tensioned to reduce the size of the first loop opening 38 as described in FIG. 3J. A tool 48 (e.g., pliers) may be used to hold the second loop segment 22 while at least a portion of the second tail end of the plug suture 22 is tensioned or pulled in order to maintain (and not reduce) the second loop opening 42 of the second looped segment 40. The reduction of the first loop opening 38 size allows a portion of the first looped segment 36 to contact a portion of the outer surface of the plug suture 22 and/or the lumen 32. At least a portion of the second tail end 26 of the plug suture 22 can be tensioned again to reduce the size of the second loop opening 42. The reduction of the second loop opening 42 size allows a portion of the second looped segment 40 to contact a portion of the outer surface of the plug suture 22, the first looped segment 36 and/or the lumen 32. Reducing both the first loop opening 38 and the second loop opening 42 creates the suture plug 4. At least a portion of the first tail end 24 and the second tail end 26 should be severed to create a compact suture plug 4.

In one embodiment, the method of making a suture retention assembly 2 comprises the steps of: obtaining a first suture, a second suture, and a plug suture, the plug suture comprises a first tail and a second tail end; piercing the first tail end through the first tail end longitudinally at least two times to create at least two looped segments proximate to each other and a lumen; extending a first or second free end of the first and second suture through the lumen of the plug suture longitudinally; extending the first and second free ends of the first and second suture through a first tail end and/or a second tail end of the plug suture to create a spliced tail; extending the spliced tail through the openings of the at least two looped segments; tensioning the remaining tail end of the plug suture to close or reduce the diameter of the at least two looped segments to form a plug, a plug wad or plug ball; trimming the first tail and second tail end. The method of making the suture retention assembly further comprises coupling the first end and the second ends of each of the first and second sutures to a suture needle. The plug, plug wad or plug ball is fixed or non-movable relative to the first and/or second suture.

In another embodiment, the method of making a suture retention assembly comprises the steps of: obtaining a first suture, a second suture, and a plug suture, the plug suture comprises a first tail and a second tail end; piercing the first tail end through the first tail end longitudinally at least two times to create at least two looped segments proximate to each other and a lumen; extending a first or second free end of the first and second suture through the lumen of the plug suture longitudinally; extending a first or second free ends of the first and second suture through a first tail end and/or a second tail end to create a spliced tail; extending the spliced tail through the openings of the at least two looped segments; tensioning the remaining tail end of the plug suture to close or reduce the diameter of the at least two looped segments to form a plug, a plug wad or plug ball; trimming the first tail and second tail end. The method of making the suture retention assembly further comprises coupling the first end and the second ends of each of the first and second sutures to a suture needle. The plug, plug wad or plug ball is fixed or non-movable relative to the first and/or second suture.

### Method of Attaching to Bone

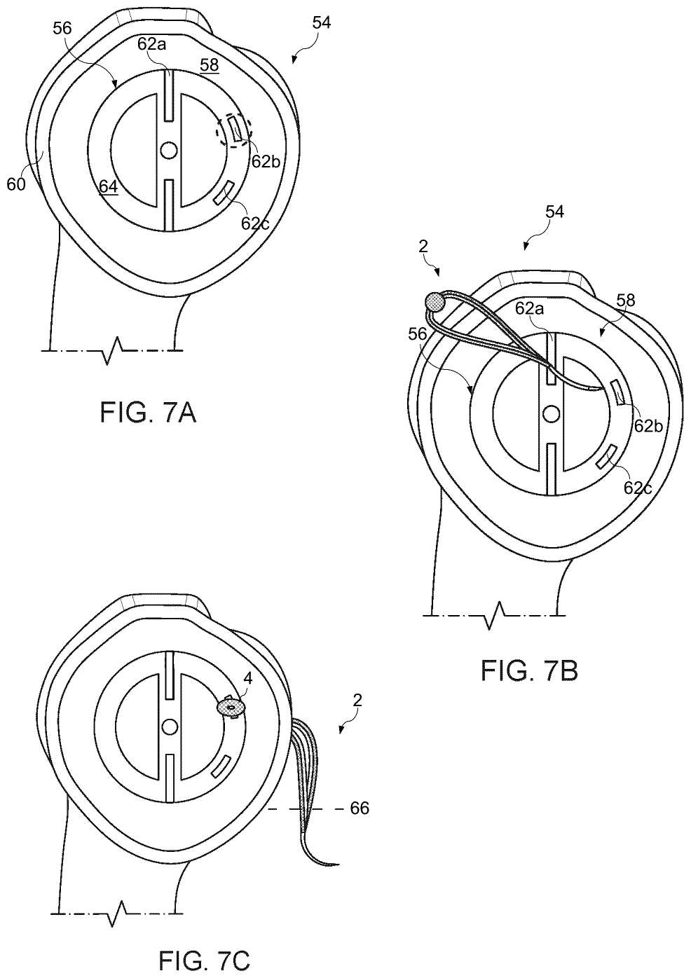

FIGS. 7A-7C, 8A-8C, 9A-9D, 10A-10D, 11A-11D and 12A-12G depict different plan views of a first embodiment of a method of using the suture retention system or kit to attach to bone and/or tissue. The bone 54 may comprise any bone within the body that can requires surgical repair. The bone 54 may comprise a first bone surface 58 and a second bone surface 60, the bone surface 54 may include an unresected or a resected surface 54. The bone 54 may comprise an implant prosthesis 56. The implant prothesis 56 may comprise one or more openings or channels 62a, 62b, 62c. The implant prosthesis 56 may be disposed onto the bone 54 and having a portion of a top surface 64 of the implant prosthesis 56 to be exposed above, flush and/or below the bone surface 54.

The surgeon may obtain one or more suture assemblies 2. The one or more suture retention assemblies 2 comprises a first suture 6, a second suture 8, a plug ball 4, and a suture needle 10. The surgeon may select the one or more openings 62a, 62b, 62c of the implant prosthesis 56 to insert, pierce or extend the suture needle 10. The surgeon may also prepare or create a pilot hole through the second bone surface 60 adjacent or next to the one or more selected openings 62a, 62b, 62c of the implant prosthesis. The second bone surface 60 may comprise the outer cortical surface into the cancellous portion (or a first bone surface 58) of the bone 54. The one or more pilot holes may be transverse to the one or more openings 62a, 62b, 62c, and/or may match or substantially the one or more openings longitudinal axis.

The surgeon may insert, pierce or extend the suture needle 10 into or through the selected one or more openings 62a, 62b, 62c of the implant prothesis 56. The suture needle 10 of the suture retention assembly 2 may extend through a cancellous portion of the bone 54 and out of the pilot hole at a first pilot hole location. The surgeon may tension the suture retention assembly 2 to ensure the first suture 6 and the second suture 8 extend fully outside the pilot hole. The tensioning of the suture retention assembly 2 allows the plug ball 4 to change shape, from a first position, the plug ball 4 at an uncompressed state, to a second position, the plug ball 4 at a compressed state. The plug ball 4 at a compressed state may comprise a plug ball width that is greater than the opening width of the one or more openings 62a, 62b, 62c. At least a portion of the plug ball 4 may be above, flush and/or below the implant prosthesis top surface 64 or the bone first surface 58.

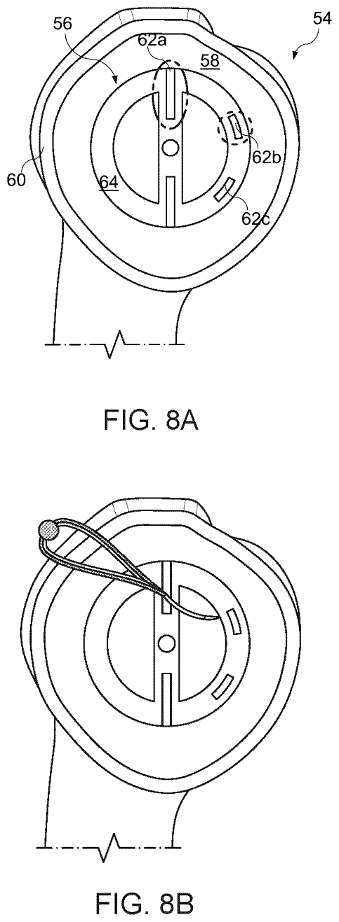

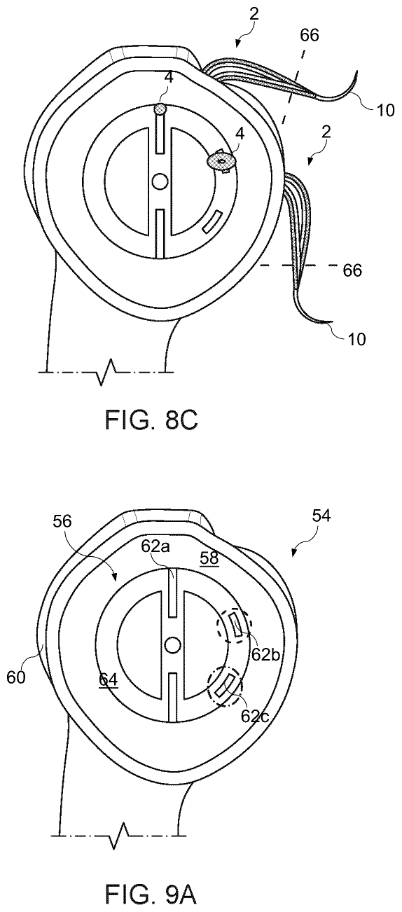

In another embodiment, a plurality of suture retention assemblies 2 may be used to further secure the implant prothesis to the bone 54 as shown in FIGS. 8A-8C, 9A-9C, and 10A-10D. In one embodiment, the implant prosthesis 54 may comprise a first opening or channel 62a having a first position, a second opening or channel 62b having a second position, and a third opening or channel 62c having a third position. Each of the first position, second position and third positions are different. Accordingly, the number of pilot holes should match the number of assemblies used to secure the suture retention assemblies 2 through the first, second and third opening 62a, 62b, 62c of the implant prosthesis 56. Accordingly, the first opening 62a at a first position may correspond to a first pilot hole at a first pilot hole location, the second opening 62b at a second position may correspond to a second pilot hole at a second pilot hole location, the third opening 62c at a third position may correspond to a third pilot hole at a third pilot hole location.

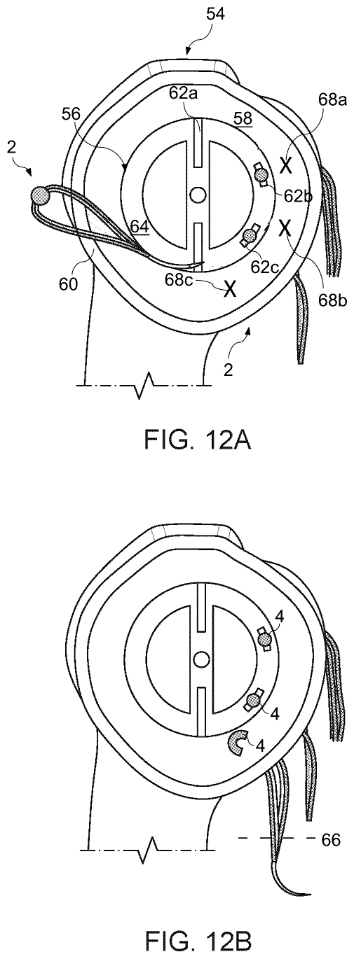

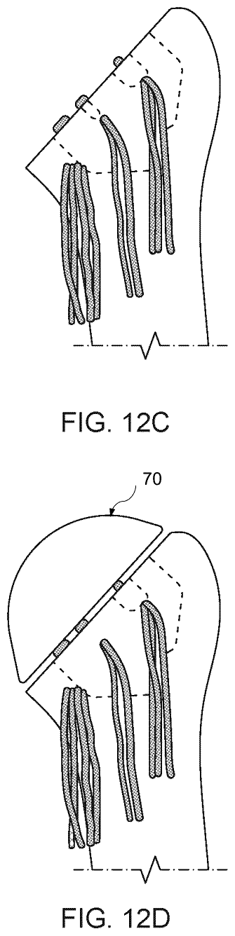

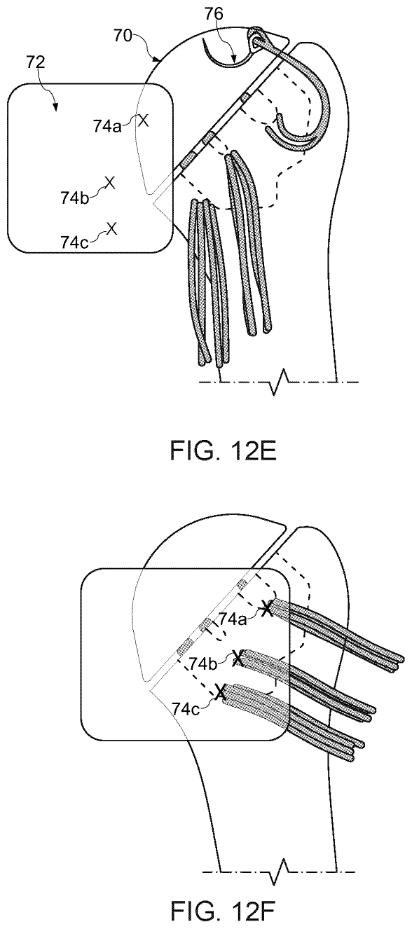

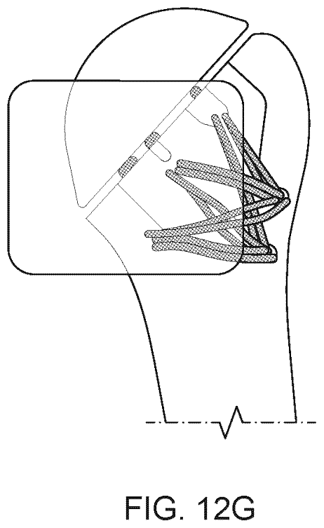

In another embodiment, the method of attaching the suture retention assembly 2 to bone may further comprise the step of: inserting, extending or piercing the needle 10 of the one or more suture retention assemblies 2 into or through a one or more locations 66a, 66b, 66c of the first bone surface 58. The first bone surface 58 may comprise a cancellous bone. Accordingly, a first suture retention assembly may be inserted, pierced or extended through a first bone surface 58 at a first location 68a, a second location 68b, a third location 68c, and/or any combination thereof as shown in FIGS. 12A-12D. Each of the first, second and third locations 68a, 68b, 68c may correspond to adjacent pilot holes through the second bone surface 60.

In another embodiment, the method of attaching one or more suture retention assemblies 2 to bone may comprise the step of reattaching a portion of tissue 72 to the bone 54. Reposition the tissue 72 to overlay a portion of the bone 54. Pierce the tissue in at least one location 74a, 74b, 74c with a tool (e.g., a snare, a shuttle or a suture passer) to create at least one tissue opening. Pull, pass or extend the one or more suture assemblies through the at least one tissue opening. The piercing of the tissue may comprise a second and/or third location with a second or third suture assemblies. Pull or tension each of the suture retention assemblies free ends 12a, 12b, 14a, 14b to ensure a portion of the a portion of the tissue approximates the bone 54.

In another embodiment, the method of use may comprise the step of severing 66 the at least one suture needle 10 from the first suture retention assembly 2. This allows access to the first free ends and the second free ends of each of the suture retention assemblies. In one embodiment, the severing 66 of the suture needles may occur prior to the step of reattaching the portion of the tissue to the bone or after reattaching the portion of the tissue to the bone. If the suture needles 10 are still attached to the suture retention assemblies 2, the suture needles may pierce the tissue in at least one tissue location 74a, 74b, 74c.

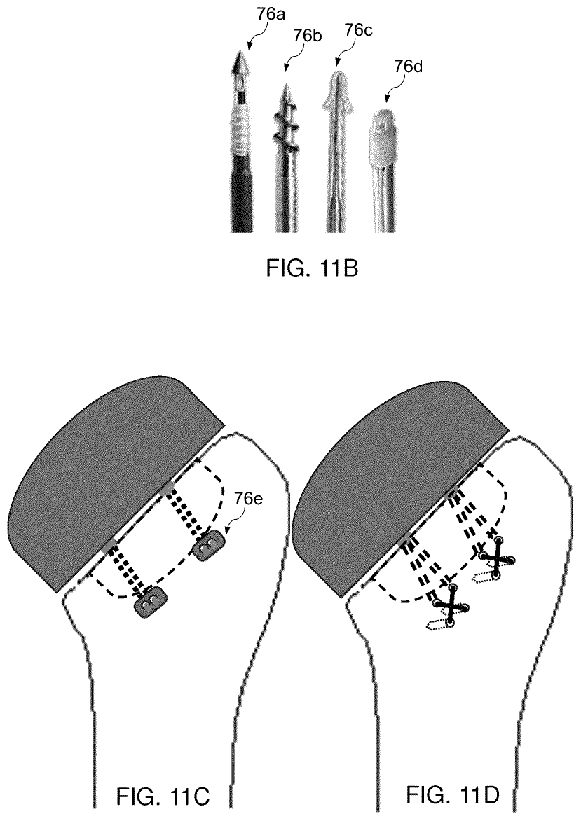

In another embodiment, the method of attaching at least one suture retention assembly 2 to the bone may comprise the step of securing or and/or fixate the one or more suture retention assembly 2 to the bone with one or more anchoring elements 76a, 76b, 76c, 76d, 76e as shown in FIGS. 11A-11D and 12G. The fixation may comprise a knotted or knotless fixation technique. The fixation step may further comprise tensioning each of the first and second suture retention assemblies to allow the plug ball 4 to be movable from an uncompressed state to a compressed state. The compressed state allows the plug ball to collapse axially and expand radially with a compressed radial diameter that is larger than the hole, opening or channel which it was introduced (whether it's disposed on the implant prosthesis or a portion of the bone) preventing or restricting the compressed or collapsed plug ball 4 from going through the opening or pilot holes to achieve the anchoring or retention required. Alternatively, the plug or plug ball 4 may comprise a uniform shape that does not change from an uncompressed state to a compressed state. At least a portion of the plug ball 4 may sit above or site below the resected surface or top surface of the implant prosthesis.

### Example Embodiments

A suture retention system or kit comprising: a first and a second suture, the first and second sutures including a first free end and a second free end; a suture plug ball, the at least a portion of the suture plug ball comprising a lumen, the first free ends of the first and second sutures extend through the lumen longitudinally, the suture plug ball disposed between the first free end and the second free end of the first and second sutures; and a suture needle, the suture needle comprises a needle point end and a swaged end, the first free ends and the second free ends of the first and second suture coupled to the swaged end of the suture needle.

The suture plug ball is movable relative to the first and second sutures. The system or kit further comprises an anchoring element. The anchoring element comprises a flexible, a rigid anchor or a button. The flexible anchor comprises a textile suture anchor. The first suture or second suture comprise a flat suture, a round suture, a flat-to-round suture, and/or any combination thereof. The first suture and second suture comprise a flat suture, a round suture, a flat-to-round suture, and/or any combination thereof. The first suture comprises a first suture material, the second suture comprises a second suture material, and the suture plug ball comprises a plug material, the plug material is different than the first suture material and the second suture material.

The method of making a suture retention kit or system comprises the steps of: obtaining a first suture, a second suture, and a plug suture, the plug suture comprises a first tail and a second tail end; piercing the first tail end through the first tail end longitudinally at least two times to create at least two looped segments having at least two openings proximate to each other and a lumen; extending a first free ends of the first and second suture through the lumen of the plug suture longitudinally; extending the first free ends or second free ends through the at least two openings of the at least two looped segments; tensioning the first tail end and/or second tail end of the plug suture to close or reduce the diameter of the at least two looped segments to form a plug, a plug wad or plug ball; and coupling the first free ends and the second free ends of each of the first and second sutures to a suture needle.

The method of making a suture retention kit or system comprises the steps of: obtaining a first suture, a second suture, and a plug suture, the plug suture comprises a first tail and a second tail end; piercing the first tail end through the first tail end longitudinally at least two times to create at least two looped segments having at least two openings proximate to each other and a lumen; extending a first free ends or second free ends of the first and second suture through the lumen of the plug suture longitudinally; extending a first free ends or second free ends of the first and second suture through the first tail end of the plug suture to create a spliced tail portion; extending the spliced tail portion through the at least two openings of the at least two looped segments; tensioning the first tail end and/or second tail end of the plug suture to close or reduce the diameter of the at least two looped segments to form a plug, a plug wad or plug ball; and coupling the first free ends and the second free ends of each of the first and second sutures to a suture needle.

The method of using the suture retention system or kit comprises the steps of: securing an implant prosthesis to a resected surface of a bone, the implant prosthesis having a top surface and one or more openings or channels, the top surface may be flush or below the resected surface of the bone; obtaining at first suture retention assembly and a second suture retention assembly, each of the first and second retention suture assemblies comprise a first suture, a second suture, a plug ball, and a suture needle; preparing or creating a first and second pilot hole through the outer surface (cortical surface) into the cancellous portion of the bone; inserting the first suture needle of the first suture retention assembly through the opening or channel disposed on the top surface of the implant prosthesis and the first pilot hole; inserting the second suture needle of the second suture retention assembly through a portion of the resected surface of the bone and the second pilot hole; reattaching a portion of tissue to the bone; severing the first suture needle from the first suture retention assembly and the second suture needle from the second suture retention assembly; and fixating the first free end and the second free end of the first and second suture to a portion of the bone with at least one anchoring element.

The bone comprises the humerus and the tissue comprises the subscapularis. The step of reattaching a portion of tissue to the bone comprises extending the first suture needle through a first location and the second suture needle through a second location on the portion of the tissue. The step of reattaching a portion of the tissue to the bone comprises snaring the first free end and the second free ends of the first suture retention assembly and the second free end of the suture assembly through a first location and a second location on a portion of the tissue. The snaring comprises a shuttle loop, a snare, a needle snare or suture passer. The step of fixating comprises the step of creating one or more bone holes. The implant prosthesis comprises a stemless humeral implant. The step of fixating comprises knotted fixation or a knotless fixation.

The method of using the suture retention system comprises the steps of: securing an implant prosthesis to a resected surface of a bone, the implant prosthesis having a top surface and one or more openings or channels, the top surface may be flush or below the resected surface of the bone; obtaining at first suture retention assembly and a second suture retention assembly, each of the first and second retention suture assemblies comprise a first suture, a second suture, a plug ball, and a suture needle; preparing or creating a first and second pilot hole through the outer surface (cortical surface) into the cancellous portion of the bone; inserting the first suture needle of the first suture retention assembly through the opening or channel disposed on the top surface of the implant prosthesis and the first pilot hole; inserting the second suture needle of the second suture retention assembly through a portion of the resected surface of the bone and the second pilot hole; reattaching a portion of tissue to the bone; severing the first suture needle from the first suture retention assembly and the second suture needle from the second suture retention assembly; and fixating the first free ends and the second free ends of the first and second suture retention assemblies to a portion of the bone with one or more anchoring elements.

The method of using the suture retention system or kit comprises the steps of: securing an implant prosthesis to a resected surface of a bone, the implant prosthesis having a top surface and one or more openings or channels, the top surface may be flush or below the resected surface of the bone; obtaining at least one suture retention assembly, the at least one suture retention assembly comprises a first suture, a second suture, a plug ball, and a suture needle; preparing or creating a pilot hole through the outer surface (cortical surface) into the cancellous portion of the bone; inserting the suture needle through the opening or channel disposed on the top surface of the implant prosthesis and the pilot hole; severing the suture needle from the first and second sutures to access a first free end and a second free end of the first and second suture; and fixating the first free end and the second free end of the first and second suture to a portion of the bone with at least one anchoring element.

I/We claim:

1. The method of making a suture retention system comprising the steps of:
obtaining a first suture, a second suture, and a plug suture, the plug suture comprises a first tail and a second tail end, the first and second sutures including a first free end and a second free end;
piercing the first tail end of the plug suture longitudinally with the second tail end of the plug suture at least two times to create a lumen, a first looped segment having a first looped opening and a second looped segment having a second looped opening, the first looped segment and the second looped segment proximate to each other;
extending the first free ends or the second free ends of the first suture and second suture through the lumen of the plug suture;
tensioning the second tail end of the plug suture to have the first looped segment and the second looped segment contact an outer surface of the lumen of the plug suture to form a plug ball;
coupling the first free ends and the second free ends of each of the first and second sutures to a suture needle.

2. The suture retention system of claim 1, wherein the plug ball is movable relative to the first and second sutures.

3. The suture retention system of claim 1, wherein the system further comprises one or more anchoring elements.

4. The suture retention system of claim 1, wherein the one or more anchoring elements comprises flexible anchor, a rigid anchor or a button.

5. The suture retention system of claim 1, wherein the first suture comprises a flat suture, a round suture, a flat-to-round suture, and any combination thereof.

6. The suture retention system of claim 1, wherein the second suture comprises a flat suture, a round suture, a flat-to-round suture, and/or any combination thereof.

7. The method of making a suture retention system comprises the steps of:
obtaining a first suture, a second suture, and a plug suture, the plug suture comprises a first tail and a second tail end, the first and second sutures including a first free end and a second free end;
piercing the first tail end of the plug suture longitudinally with the second tail end of the plug suture at least two times to create a lumen, a first looped segment having a first looped opening and a second looped segment having a second looped opening, the first looped segment and the second looped segment proximate to each other;
extending the first free ends or the second free ends of the first suture and second suture through the lumen of the plug suture;
extending the first free ends or the second free ends of the first suture and second suture through the first looped opening of the first looped segment and the second looped opening of the second looped segment;
tensioning the second tail end of the plug suture to have the first looped segment and the second looped segment contact a portion of an outer surface of the lumen of the plug suture to form a plug ball; and
coupling the first free ends and the second free ends of each of the first and second sutures to a suture needle.

8. The method of claim 7, wherein the plug ball is fixed or non-movable relative to the first and second sutures.

9. The method of claim 7, wherein the system further comprises one or more anchoring elements.

10. The method of claim 7, wherein the one or more anchoring elements comprises a flexible anchor, a rigid anchor or a button.

11. The method of claim 7, wherein the method further comprises the step of piercing the first free ends or the second free ends of the first suture and second suture through a second lumen positioned proximate to the second tail end of the plug suture.

12. The method of using the suture retention system to attach to a portion of bone comprises the steps of:
Securing an implant prosthesis a bone, the implant prosthesis having a top surface and at least one opening having a first location;
Providing at least one suture retention system, the suture retention system comprises at least one suture retention assembly and at least one anchoring element, the at least one suture retention assembly comprises a first suture, a second suture, a plug ball, and a suture needle;
Creating at least one pilot hole through a portion of the bone at a second location, the second location is different than the first location;
Extending a through the at least one opening at the first location of the implant prosthesis to exit out the at least one pilot hole;
Tensioning the first suture and second suture of the suture retention assembly to secure a portion of the plug ball into the at least one opening of the implant prosthesis;
Severing the suture needle from the first and second sutures to access a first free end and a second free end of the first and second sutures; and
Fixating the first free end and the second free end of the first and second suture to a portion of the bone with the at least one anchoring element.

13. The method of use of claim 12, wherein the method further comprises tensioning the first suture and the second suture to secure a portion of the plug ball into the one or more openings of the implant prosthesis by allowing the plug ball to change from an uncompressed state to a compressed state.

14. The method of use of claim 13, wherein the compressed state creates a plug ball diameter that is larger than a width of the one or more openings of the implant prosthesis allowing retention of the implant prosthesis against a portion of the bone.

15. The method of use of claim 12, wherein the method further comprises reattaching a portion of a tissue to the bone.

16. The method of use of claim 15, wherein the method of reattaching a portion of a tissue to the bone comprises the step of extending the first free end and the second free end of the first and second sutures of the at least one retention system through a portion of the tissue.

17. The method of use of claim 12, wherein the step of fixating comprises knotless fixation or a knotted fixation.

18. The method of use of claim 12, wherein the plug ball is movable relative to the first and second sutures or the plug ball is non-movable relative to the first and second sutures.

19. The method of use of claim 12, wherein the first suture comprises a first suture pattern and the second suture comprises a second suture pattern, the second suture pattern is different from first suture pattern.

20. The method of use of claim 12, further comprises the step of inserting a second suture assembly into a cancellous bone surface in a third location to exit out a second pilot hole at a fourth location.
